(19) United States
(12) Patent Application Publication
(10) Pub. No.: US 2026/0294242 A1
(43) Pub. Date: Oct. 1, 2026

(54) GUIDED SENSOR ALIGNMENT FOR VISUAL FUNCTION TESTING

(71) Applicant: Charles Quentin Davis, Frederick, MD (US)

(72) Inventor: Charles Quentin Davis, Frederick, MD (US)

(21) Appl. No.: 19/665,673

(22) Filed: May 1, 2026

Related U.S. Application Data
(63) Continuation of application No. PCT/US2026/020442, filed on Mar. 23, 2026.
(60) Provisional application No. 63/777,316, filed on Mar. 25, 2025.

(51) Int. Cl. A61B 3/15; A61B 3/00

(52) U.S. Cl. CPC A61B 3/152; A61B 3/0091

(57) ABSTRACT

Systems and methods are provided for physiological measurement using: a light emitter, a camera, and one or more processors programmed by executable instructions. The system may access image data from the camera. The image data may represent an eye of a patient. The system may further determine a position at which a sensor should be placed based on the image data, the position determined so as to align the sensor for measuring a signal from the eye. The system may further generate a visual guide regarding positioning of the sensor, the visual guide including an alignment zone that includes the determined position. The system may further cause the light emitter to emit light into the eye and take a physiological measurement using the sensor.

### CROSS-REFERENCE TO RELATED APPLICATIONS

This application is a continuation application of PCT Application No. PCT/US 2026/020442, filed Mar. 23, 2026, and titled “GUIDED SENSOR ALIGNMENT FOR VISUAL FUNCTION TESTING”, which claims priority to U.S. Provisional Patent Application No. 63/777,316, filed on Mar. 25, 2025 and titled “GUIDED SENSOR ALIGNMENT FOR VISUAL FUNCTION TESTING”, the contents of each of which are incorporated by reference herein and made part of this specification.

### FIELD

The present disclosure relates to the field of non-invasive patient monitoring. More specifically, the disclosure relates to sensor alignment for tests relating to visual function.

### BACKGROUND

A variety of tests can be performed to provide an indication of visual function. One such test may be electroretinography. Electroretinography can be used to measure electrical activity in the retina of a patient in response to exposure to light. To conduct an electroretinography test, sensors are placed in proximity of the patient's eye to detect the retina's electrical responses to light. The results may be analyzed, such as by a caregiver, to diagnose various conditions affecting the visual pathway.

### SUMMARY OF SOME EMBODIMENTS

In some aspects, the techniques described herein relate to a device including: a light emitter; a camera; and one or more processors programmed by executable instructions to: access image data from the camera, the image data representing an eye of a patient; determine a position at which a sensor should be placed based on the image data, the position determined so as to align the sensor for measuring a signal from the eye; generate a visual guide regarding positioning of the sensor, the visual guide including an alignment zone that includes the determined position; cause the light emitter to emit light that reaches the eye; and take measurements using the sensor, the measurements providing a physiological measurement.

In some aspects, the techniques described herein relate to a computer-implemented method including: as performed by a computing system including one or more computer processors programmed to execute specific instructions, accessing image data from a camera, the image data representing an eye of a patient; determining a position at which a sensor should be placed based on the image data, the position determined so as to align the sensor for measuring a signal from the eye; generating a visual guide regarding positioning of the sensor, the visual guide including an alignment zone including the determined position; and taking measurements using the sensor, the measurements providing a physiological measurement.

In some aspects, the techniques described herein relate to one or more non-transitory computer-readable media storing instructions that, when executed, cause a computing system to perform operations including: accessing image data from a camera, the image data representing an eye of a patient; determining a position at which a sensor should be placed based on the image data, the position determined so as to align the sensor for measuring a signal from the eye; generating a visual guide regarding positioning of the sensor, the visual guide including an alignment zone including the determined position; and taking measurements using the sensor, the measurements providing a physiological measurement.

In some aspects, the techniques described herein relate to a device providing an indication of visual system function of a patient including: a light emitter; an optical assembly arranged so that light emitted from the light emitter reaches an eye of the patient; a camera arranged to image both the eye of the patient and an intended position for a sensor adapted to be applied and removed from the patient; and a controller that modulates a light emission from the light emitter to create a light stimulus and receives and analyzes an electrical signal from a visual system of the patient via the sensor, and provides the indication of visual system function based on that analysis, wherein the device is arranged to be used by a caregiver, and wherein the controller provides a visual guide to the caregiver regarding placement of the sensor based on images from the camera.

### BRIEF DESCRIPTION OF DRAWINGS

FIG. 1A illustrates use of a physiological measurement device, in accordance with some aspects of the present disclosure.

FIG. 1B illustrates use of a physiological measurement device, in accordance with some aspects of the disclosure.

FIG. 2A illustrates placement of a sensor, in accordance with some aspects of the disclosure.

FIG. 2B illustrates alternative placements of a sensor which may be used in accordance with some aspects of the present disclosure.

FIG. 3A illustrates an example visual guide for alignment of a sensor.

FIG. 3B illustrates an example visual guide for alignment of a sensor as a patient's eye moves over time.

FIG. 4 is a flow diagram of an example method for alignment of sensor position.

FIG. 5 illustrates a general architecture for an alignment system including an arrangement of computer hardware and software that may be used to implement aspects of the present disclosure.

### DETAILED DESCRIPTION OF SOME EMBODIMENTS

Generally described, aspects of the present disclosure relate to sensor alignment in connection with use of physiological measurement devices, such as devices that perform tests relating to visual function or other aspects of ocular health. One such test may be an electroretinography (ERG) test. ERG testing can provide a detailed assessment of a patient's ocular condition. Data from this assessment may be used in diagnosing a variety of ocular conditions, which may improve health outcomes. Aspects of the present disclosure generally relate to improving the effectiveness of testing devices by using an alignment system and method that facilitate alignment of sensors measuring data during testing, thereby improving the quality of the test data generated by the testing devices (e.g., by providing higher signal to noise ratio or “SNR,” by improving test to test reliability through a reduction in variations caused by sensor positioning, etc.). Improvements to the quality of the data may accordingly improve diagnosis of ocular conditions (e.g., by reducing false positives or false negatives that may be caused by poor quality data).

ERG testing exposes a retina of the patient to stimuli emitted from a light source. The light source may, for example, emit light in a series of pulses forming a pattern. Sensors placed in proximity to the patient's eye may be used to help measure the retina's response to the stimuli. For example, the sensors may include adhesive components designed to adhere to the patient's skin near the patient's eye and electrodes designed to electrically connect a region of the patient to measuring equipment. The measuring equipment can be part of the sensor, where digitized data can be transmitted (via wired or wireless connection) to a unit that emits the light or otherwise performs the test. Alternatively, the measuring equipment can be separate from the sensor (e.g., part of the unit) with analog signals passing between the sensor and the measuring equipment. Data from the sensors may, for example, be represented as a waveform. A caregiver (e.g., a health-care professional, a doctor, a nurse, etc.) may analyze the data to help diagnose the patient. A caregiver may, for example, evaluate electrical signals detected by the sensors during ERG to diagnose ocular conditions.

Electrical signals may also be used to detect electric fields from the retinal pigment epithelium. For example, the electrooculogram (EOG) can be measured as well as the c-wave from the ERG response to light stimulus. The position of the eye and changes therein can also be estimated via electrical signals. The electromyogram (EMG) can also be measured via electric signals, for example measuring the occurrence of blinks, squinting, and the like.

As an alternative to detecting an electrical signal, the sensor may detect other types of signals. The sensor may, for example, detect at least one of an optical signal, a thermal signal, a mechanical signal, or a magnetic signal. A mechanical signal may, for example, be associated with eyelid blinking or squinting. These mechanical signals may be, as an example, a result of uncomfortably bright lights and thereby be a measure of photophobia. Photophobia may be caused, for example, by traumatic brain injury, head injuries, migraines, eye conditions, allergic reactions, certain medications, and stress.

Ocular conditions diagnosed using ERG may include, but are not limited to, diabetic retinopathy, glaucoma, retinitis pigmentosa, macular degeneration, other conditions affecting the visual pathway (e.g., schizophrenia), and the like. These conditions may lead to vision loss. Accurate diagnosis may improve health outcomes for patients. However, accurate diagnosis may be adversely impacted by incorrect sensor alignment during ERG testing of the patient.

As one example, a misaligned sensor may distort data received during testing (e.g., by changing the amplitude or timing of the response). This may increase difficulty for a caregiver in interpreting waveforms representing the data. As another example, a misaligned sensor may miss responses to stimuli produced by the retina during testing. This may result in missed diagnosis of conditions by a caregiver. Inaccurate diagnosis, including missed diagnosis, may delay proper treatment, increasing the likelihood of negative health outcomes for the patient, such as vision loss. Misaligned sensors may, as a further example, decrease reliability between tests. With respect to ERG testing, as one example, multiple tests may be performed on a patient to verify accuracy of results or to see if the patient's condition is changing over time. The results may, however, vary based on sensor placement. For example, sensor placement may change the amplitude of an electrical signal received by the sensor during placement. If sensor alignment differs significantly between tests, the reliability of the measurements may suffer. This may adversely impact a caregiver's ability to diagnose a patient based on the testing, increasing the likelihood of negative health outcomes of the patient.

Accurate sensor alignment may depend on features of the eye that may change over short periods of time. As a patient's eye moves, for example, features of eye components—such as positions of the eyelids, position of the iris, position of the pupil, position of a canthus (i.e., a corner) of the eye, and the like, or some combination thereof—can change. This increases the difficulty for caregivers in properly aligning the sensors for testing. Moreover, a relatively small variance in the alignment may significantly decrease the quality of sensor data and effectiveness of the physiological measurement device.

Aspects of the present disclosure address some or all of the issues noted above, among others, using an alignment system for physiological measurement devices. The alignment system intakes image data from a camera directed towards a patient's eye. From this image data, the alignment system may determine features, such as positions of one or more components of the patient's eye. Components of the eye may include, but are not limited to, a superior eyelid (also referred to as an upper eyelid), an inferior eyelid (also referred to as a lower eyelid), an iris, a pupil, a canthus of the eye, and the like, or some combination thereof. In some embodiments, other facial components and features may be detected, such as the position of the patient's nose or eyebrow.

As one example, the alignment system may detect a pupil of an eye. The alignment system may use a computer vision algorithm such as, for example, a machine learning based algorithm, to identify the pupil. Once the alignment system identifies the pupil, the alignment system may determine a position, with respect to the pupil, at which the sensor is to be placed. For example, the alignment system may determine that the sensor or a particular portion thereof (e.g., an edge that is proximate to the patient's nose) is to be positioned inferior to a center point of the pupil (or inferior to an inferior edge of the pupil), and within a medial and/or lateral offset (e.g., defined in millimeters) of the center point. As another example, the alignment system may determine that the sensor is to be offset inferior to the bottom of the pupil or eyelid by less than a maximum distance (e.g., defined in millimeters). The alignment system may, for example, determine that the sensor is to be offset inferior to the bottom of the pupil or eyelid by a certain distance. The alignment system may subsequently generate and present an indication of the proper position of the sensor. In addition to the indication of the proper position of the sensor, the alignment system may generate dynamic feedback regarding the position during or after placement of the sensor. The indication and dynamic feedback may improve a caregiver's ability to place the sensor in a properly aligned position. The indication and dynamic feedback may additionally, or alternatively, improve a caregiver's ability to determine if a previously placed sensor is in a properly aligned position.

The indication and/or dynamic feedback regarding positioning of the sensor may include, for example, a visual guide presented through a display of the physiological measurement device. Although one position may be the preferred location for measurement, the visual guide may comprise an alignment zone that is a region of acceptable locations that includes the preferred location and other locations having acceptable performance. The display may include an image of the patient's eye and surrounding facial features, as the patient's eye moves (e.g., over a short period of time during sensor placement prior to commencement of a test, after the sensor is placed but prior to commencement of a test, etc.). The visual guide may include a graphical alignment zone that automatically changes position responsive to movement of the patient's eye. The alignment system may, for example, superimpose the alignment zone over a region of the display corresponding to the determined position for the sensor. The alignment system may superimpose the alignment zone in a band in the superior-inferior direction that includes the center of the pupil, for example. The alignment zone may, in some examples, be limited in extent in a medial-lateral direction to a span that includes a center of the pupil. The alignment system may additionally, or alternatively, superimpose alignment zone in a medial-lateral direction offset from the lower eyelid of the eye. The alignment system may, in some examples, limit the alignment zone to extend in the medial-lateral direction to a first span that includes the center of the pupil and is limited in extend in the superior-inferior direction to a second span that includes an offset from the lower eyelid. As one example, the alignment zone may be a band indicating a 1-dimensional limitation in acceptable sensor locations. Other options are also possible. The alignment zone may, for example, be other 2-dimensional shapes indicating a multidimensional limitation in acceptable sensor locations, such as a rectangle, oval, or the like.

The visual guide may allow a caregiver to track accurate placement over time as they are positioning the sensor. This represents an improvement to visual system testing at least because it provides a simplified method of highlighting a recommended position for the sensor which may improve alignment of the sensor for visual system testing. This improves the ability of the caregiver to properly position the sensor while taking into account the movements of the patient's eye.

The visual guide may allow a caregiver to assess the quality of placement of a sensor prior to test completion. This represents an improvement to visual system testing at least because it provides a feedback mechanism where the caregiver can assess and possibly correct sensor alignment prior to test completion, thereby reducing positioning-induced errors. Sensors may be adapted to be applied to, removed from, and reapplied to the patient in order to correct poorly aligned sensor placements.

The visual guide may, additionally, or alternatively, include one or more misalignment zones. A misalignment zone may indicate a region of the image where the sensor would be misaligned if positioned in the corresponding portion of the patient's face. The alignment system may, for example, superimpose the misalignment zone over a region of the display corresponding to a position where the sensor would be misaligned if positioned on the patient, such as positioning an edge of the sensor at a location that is medial to the patient's pupil and lateral to the patient's nose. Having both an alignment zone and a misalignment zone may be advantageous in that it may provide additional feedback to placement or assessment of the placement of the sensor.

The visual guide may, in further examples, account for the position of more than one component of the patient's eye or face when determining a position for the sensor. The alignment system may, for example, identify facial components such as the pupil, iris, canthi of the eye, upper eyelid, lower eyelid, nose, eyebrows, and the like. Based on a combination of facial component positions or other features, the alignment system may further refine its determination of a position for the sensor based on the combination of facial component positions or other features. The alignment system may, for example, superimpose an alignment zone in the display. The sensor, if placed on the region of the patient's face corresponding to the alignment zone, may have improved alignment for visual system testing. The alignment system may, additionally, or alternatively, superimpose one or more misalignment zones on the display based on the combination of facial component positions or other features. The misalignment zones may identify regions of the patient's face represented in the display when the sensor would likely be misaligned, if placed.

The visual guide from the alignment system, as described above, may be used by the caregiver to improve placement of the sensor. The caregiver may then take measurements using the sensor during visual system testing. The measurements may be improved as a result of improved positioning of the sensor. This may improve diagnosis of the patient and accordingly improve health outcomes for the patient. A risk of vision loss for the patient may be reduced, for example.

Image data captured during or after placement of the sensor may represent at least part of the sensor in addition to the eye. The physiological measurement device may verify that the sensor has been positioned in the alignment zone. This additional verification operates on top of generating the visual guide regarding positioning of the sensor, so that the alignment zone is not merely displayed but is also used to check whether the sensor placement corresponds to the determined position. Improperly aligned sensors may be indicated, such as by altering presentation of the alignment zone or by presenting other feedback (e.g., visual or audio feedback). As a result, the verification can reduce instances in which measurements providing a physiological measurement taken with the sensor outside the alignment zone. This supports more consistent positioning of the sensor relative to the eye of a patient, improves the quality of the signal from the eye that is measured, and thereby improves the reliability of the physiological measurement. In some examples, the caregiver may be blocked from proceeding until the sensor is repositioned correctly.

The above-described aspects and other aspects of the disclosure will now be described with regard to certain examples, embodiments, and aspects, which are intended to illustrate, but not limit, the disclosure. The foregoing aspects and many of the attendant advantages of this disclosure will become more readily appreciated as the same become better understood by reference to the following description, when taken in conjunction with the accompanying drawings. Any feature used in any embodiment described herein may be used in any combination with any other feature or in any other embodiment, without limitation.

### Example Measurement Environment

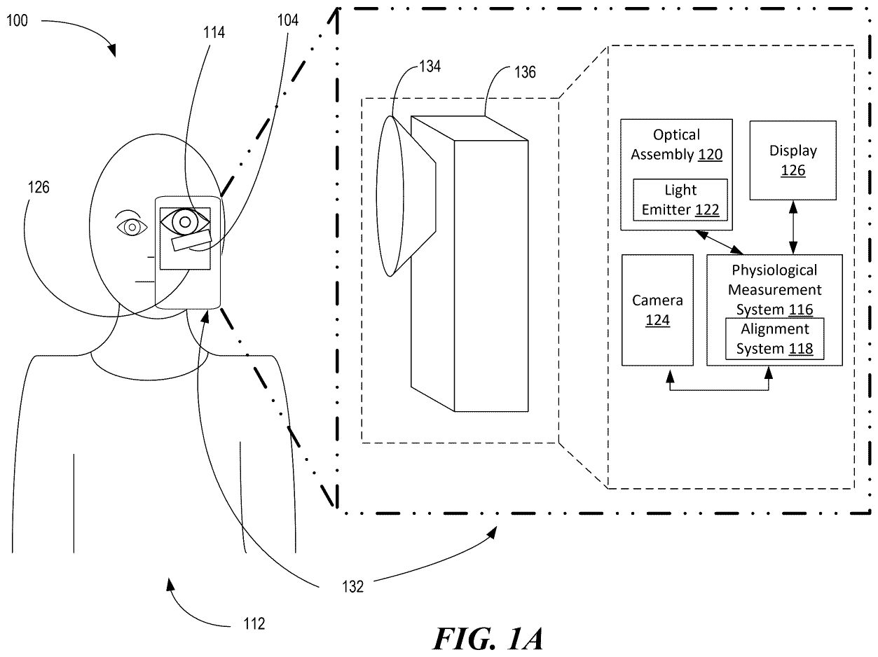

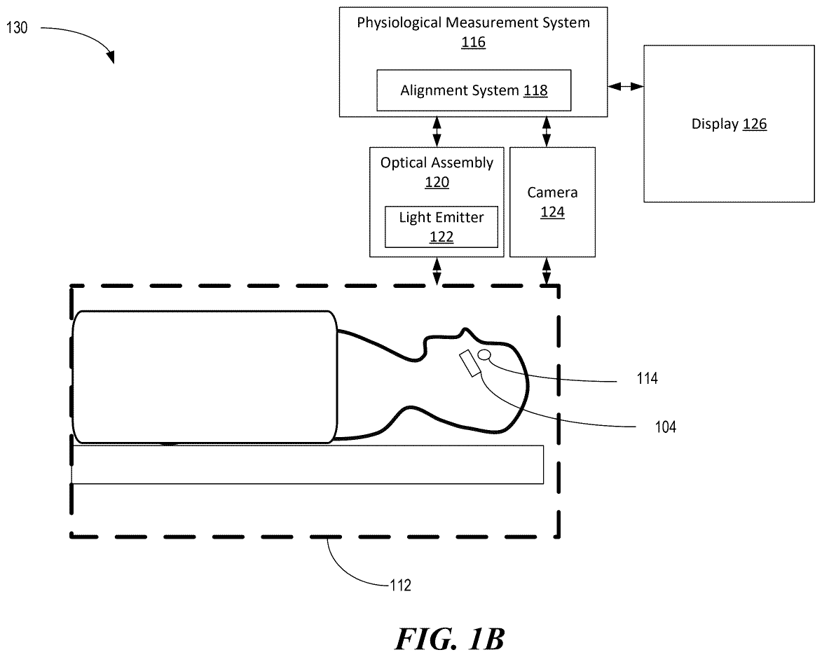

FIG. 1A illustrates an example measurement environment 100, in accordance with some aspects of the disclosure. Patient 112, as illustrated, is in an upright position. Patient 112 may, for example, be sitting or standing. Although patient 112 is illustrated as a human, the systems and methods of the present disclosure may be used with nonhuman patients, such as other mammals.

Physiological measurement device 132 ((also referred to herein simply as a “device” for brevity) may determine an alignment for sensor 104 based on image data from a camera 124. The image data, as illustrated, includes image data of eye 114 of patient 112. Device 132 may further provide a visual guide to align sensor 104 on patient 112 with respect to one or more components of eye 114. Device 132 may, for example, provide a visual guide through display 126, as further described herein.

Device 132, as illustrated, includes display 126, ocular enclosure 134 and device housing 136. Device 132, as illustrated, further includes physiological measurement system 116, optical assembly 120, and camera 124.

Physiological measurement system 116 may, in some examples, be electrically connected to optical assembly 120, camera 124, and display 126. Physiological measurement system 116 may, in some examples, cause camera 124 to capture image data with respect to patient 112. Physiological measurement system 116 may further use alignment system 118 to identify a feature of an eye 114 of patient 112. Physiological measurement system 116 may further use alignment system 118 to determine a position for a sensor 104 on patient 112. The determined position may be based on the identified feature. Physiological measurement system 116 may further output a visual guide for the determined position through display 126. This may advantageously improve positioning of sensor 104 for capturing data with respect to the eye 114 of patient 112. A caregiver may, for example, position sensor 104 onto patient 112 based on the visual guide. Measurements (e.g., ERG measurements) obtained from patient 112 may advantageously be improved based on the positioning of sensor 104 based on the visual guide from alignment system 118.

Camera 124 may be any suitable image capture device. Camera 124 may, in some examples, comprise one or more lenses, optical sensors, or other image capture components. Camera 124 may capture image data based on instructions from physiological measurement system 116. Camera 124 may capture images continuously, intermittently at regular intervals, intermittently at variable intervals, and the like, or some combination thereof. Camera 124 may, as illustrated, generate image data of patient 112. Camera 124 may, for example, generate image data including an image of an eye 114 of patient 112, surrounding facial features (e.g., nose, eyebrow, etc.) of patient 112, and the like, or some combination thereof. Camera 124 may provide image data to physiological measurement system 116.

Alignment system 118, as illustrated, is implemented by physiological measurement system 116. Alignment system 118 may analyze image data from camera 124. Alignment system 118 may, for example, determine features of eye 114 of patient 112, facial features surrounding the eye 114 of patient 112, and the like, or some combination thereof. Alignment system 118 may, for example, use one or more machine learning or computer-vision algorithms to identify features of the eye 114 of patient 112, facial features surrounding the eye of patient 112, and the like, or some combination thereof.

As one example, the alignment system 118 may detect a pupil of the eye 114. The alignment system 118 may, for example, use one or more machine learning or computer vision algorithms to identify the pupil. The one or more machine learning algorithms may include, but are not limited to, a convolutional neural network (CNN), a generative adversarial network (GAN), an autoencoder, a recurrent neural network (RNN), a transformer, support vector machines (SVMs), K-means clustering, random forests, and the like, or some combination thereof. Alternatively, computer vision methods can be employed such as the Hough transform, blob detection in binary images, erosion/dilation, machine learning based methods, and the like. Once the alignment system 118 identifies the pupil, the alignment system 118 may determine a positioning for the sensor 104 with respect to the eye 114. The alignment system 118 may subsequently generate a visual guide for the caregiver regarding the positioning of the sensor 104. This may improve a caregiver's ability to place and/or assess placement of the sensor 104 in a properly aligned position with respect to the eye 114.

A visual guide regarding positioning of the sensor 104 may, for example, be presented through display 126. The display 126 may, in some examples, include an image of the eye 114 and surrounding facial features. In some examples, display 126 may provide a substantially real time view of the eye that dynamically changes as the eye 114 moves over time.

As used herein, the term “real time” is used according to its usual and customary meaning in computing and networking and refers to the effectively contemporaneous nature of the events being described. In computing environments, different events within a single computing device (or events in different locations on a network) rarely occur at exactly the same time. There may be an offset in timing due to latencies inherent in communications, computer processing, and the like. Thus, the term “real time” does not necessarily equate to “exactly the same time,” but rather the observed effect of two or more events occurring at approximately the same time for practical purposes and when factoring in communication and processing. Thus, the concept of “real time” is often referred to herein as “substantially real time.” Delays for real-time systems may, as one example, be less than one second. The refresh rate may, as one example, be at least once per second (1 Hz).

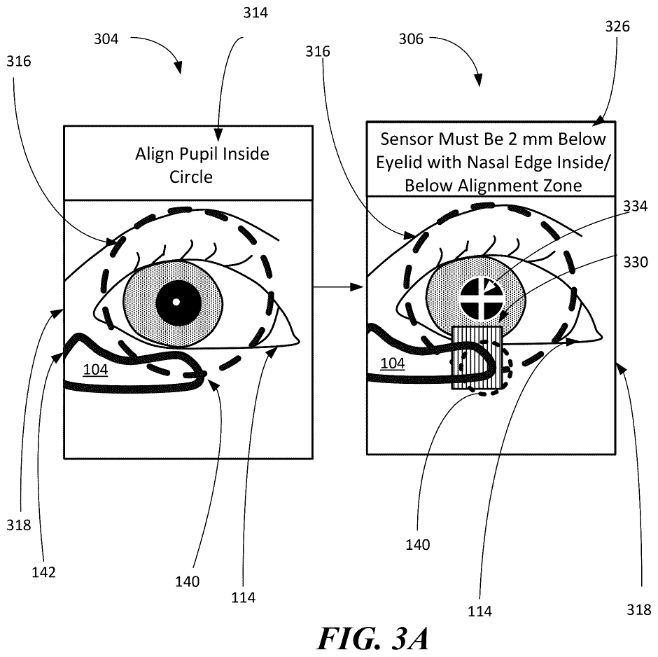

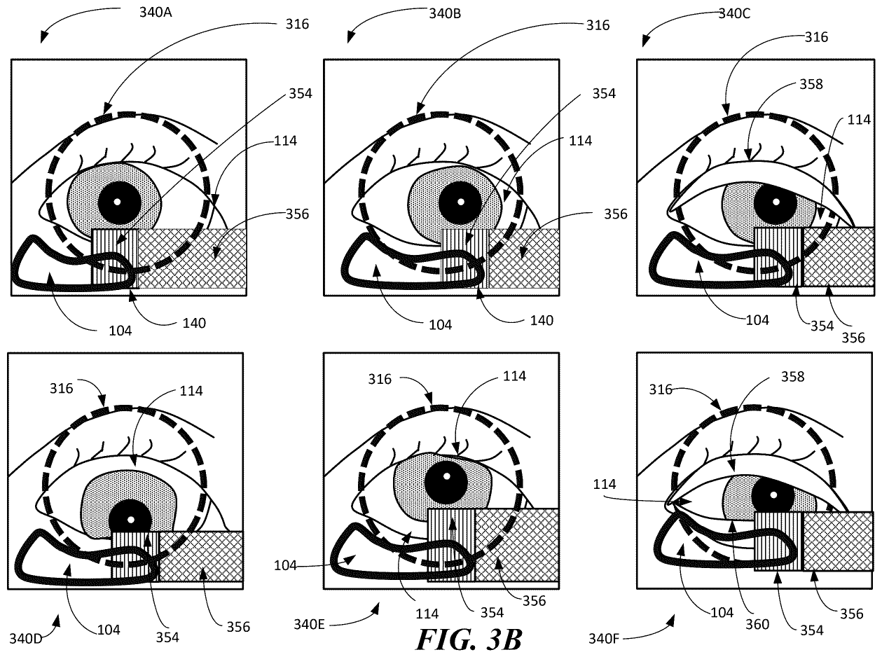

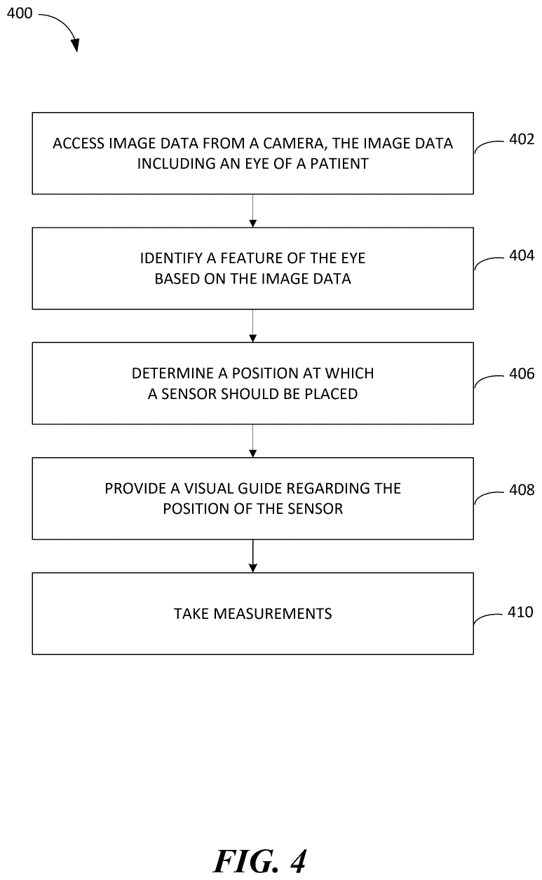

The visual guide may include a graphical alignment zone changing position responsive to movement of the patient's eye. The alignment zone may include an indication to the caregiver of the position for the sensor determined by the alignment system. The alignment system 118 may, for example, superimpose the alignment zone over a region of the display corresponding to the determined position for placement of the sensor. The alignment system 118 may superimpose the alignment zone in a band limited in the superior-inferior direction that includes the center of the pupil, for example. The alignment system 118 may additionally, or alternatively, superimpose alignment zone in a medial-lateral direction offset from the lower eyelid of the eye. The alignment system 118 may, in some examples, limit the alignment zone to extend in the medial-lateral direction to a first span that includes the center of the pupil and limit the alignment zone to extend in the superior-inferior direction to a second span that includes an offset from the lower eyelid. This may allow a caregiver to track accurate placement over time as they are placing the sensor 104, or to assess alignment quality in a previously placed sensor 104. Generating the visual guide based on movement of the eye 114 over time will be described further herein, at least with respect to FIGS. 3A-3B and FIG. 4.

The visual guide may, additionally, or alternatively, include one or more misalignment zones. A misalignment zone may indicate a region of the image where the sensor 104 would be misaligned if placed in the corresponding portion of the face of patient 112. The alignment system 118 may, for example, superimpose the misalignment zone over a region of the display corresponding to a position where the sensor 104 would be misaligned, if placed on the patient.

The visual guide may, in further examples, account for the position of more than one feature of the eye 114 when determining a position for the sensor 104. The alignment system 118 may, for example, identify features, not limited to, the pupil, iris, canthi of the eye, upper eyelid, lower eyelid, nose, eyebrows, and the like. Based on a combination of features, the alignment system 118 may further refine its determination of a position for the sensor 104. The alignment system 118 may, for example, superimpose an alignment zone in the display. The sensor 104, if placed on the region of the face of patient 112 corresponding to the alignment zone, may have improved alignment for visual system testing. Generating the visual guide based on movement of the eye 114 over time will be described further herein, at least with respect to FIGS. 3A-3B and FIG. 4.

Alignment system 118, as illustrated, is implemented as a component of physiological measurement system 116. However, other configurations may be implemented. Alignment system 118 may, as another example, be implemented on one or more computing devices separate from physiological measurement system 116. Physiological measurement system 116 may, in further examples, communicate image data from camera 124 to alignment system 118. Alignment system 118 may analyze the data using any of the methods described above to generate the visual guide relating to alignment of sensor 104.

Sensor 104 may be any suitable type of sensor. Sensor 104 may be used to detect signals, such as electrical, magnetic, optical, thermal, or mechanical signals. Sensor 104 may, as one example, be used to detect electrical signals. Sensor 104 may, for example, include a substrate, one or more electrodes, and the like, or some combination thereof. Sensor 104 may, for example, include a flexible substrate. The flexible substrate may, for example, conform to the face of patient 112. The flexible substrate may, as another example, conform to a surface of eye 114. However, other materials or configurations may be used. Sensor 104 may be rigid, semi-rigid, flexible, or some combination thereof.

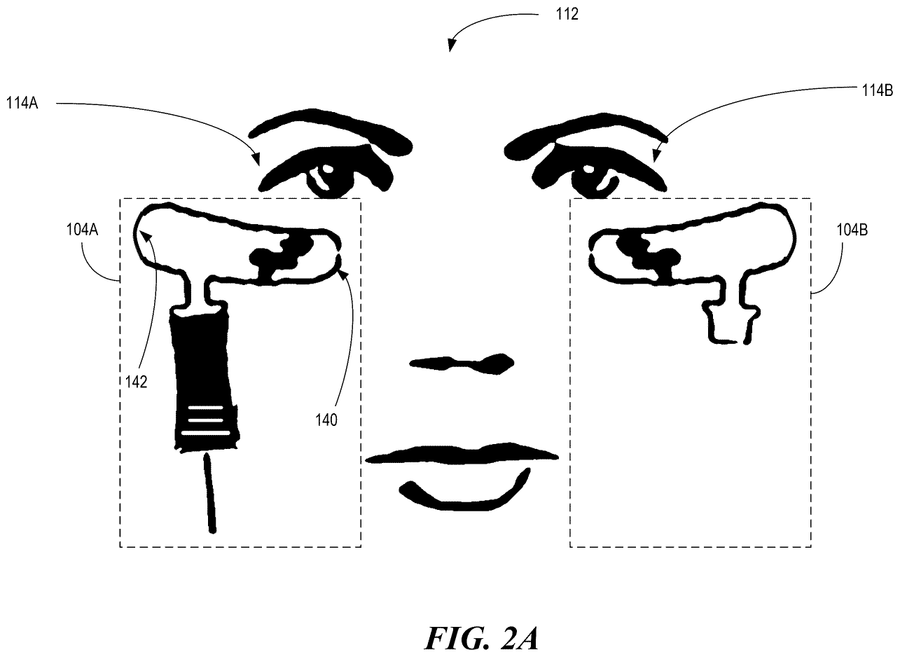

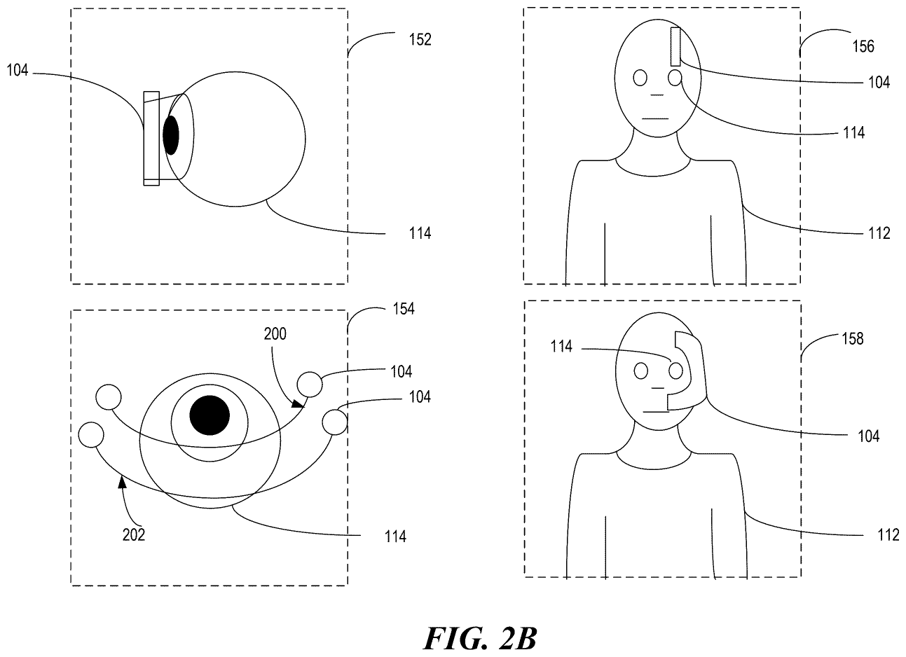

One or more detectors may be mounted onto sensor 104. The detectors may be configured to capture electrical signals responsive to testing (“electrical responses” or “responses”, for brevity) from a retina of eye 114. The detectors may, for example, be one or more electrodes positioned on sensor 104 on the surface in contact with patient 112. The one or more electrodes may be of any suitable material including, but not limited to, copper-based electrodes, silver-based electrodes, gold-based electrodes, platinum-based electrodes, iridium -based electrodes, steel-based electrodes, carbon-based electrodes, conductive polymer-based electrodes, and the like, or some combination thereof. The one or more electrodes may, for example, comprise carbon, silver, silver/silver chloride, gold, platinum, iridium, or steel. While sensor 104, as illustrated, is positioned beneath eye 114, other positions may be used. Sensor 104 may, for example, be placed on eye 114, above eye 114, at different orientations around eye 114, and the like, or some combination thereof. Various example positions of sensor 104 will be described further herein, at least with respect to FIGS. 2A-2B.

Sensor 104 may include any of the features or functions described with respect to U.S. Pat. No. 9,492,098, filed Jan. 28, 2014, entitled “Visual Electrophysiology Device” and incorporated by reference herein in its entirety. Sensor 104 may include any of the features or functions described with respect to U.S. Pat. No. 9,931,032, filed Mar. 13, 2015, entitled “System and Method for Retinopathy Detection” and incorporated by reference herein in its entirety. Sensor 104 may include any of the features or functions described with respect to U.S. Pat. No. 10,010,261, filed Nov. 9, 2016, entitled “Electrode Arrays” and incorporated by reference herein in its entirety. Sensor 104 may include any of the features or functions described with respect to U.S. Pat. No. 9,510,762, filed Sep. 4, 2013, entitled “Electrode Arrays” and incorporated by reference herein in its entirety.

Optical assembly 120, as illustrated, includes a light emitter 122. The physiological measurement system 116 may use optical assembly 120 to conduct visual system testing on patient 112. A caregiver may, for example, initiate ERG testing after positioning sensor 104. Physiological measurement system 116 may, after initiation, provide instructions to optical assembly 120 to emit light using light emitter 122. Light emitter 122 may be any suitable light emitting device. Light emitter 122 may include, for example, one or more light emitting diodes (LEDs).

Based on the instructions, optical assembly 120 may emit light with light emitter 122. Optical assembly 120 may, for example, cause emission of light into eye 114 as stimuli to obtain one or more responses from the retina of eye 114. Light directed into eye 114 may be direct or indirect illumination. Light emitter 122 may emit one or more pulses into eye 114, for example. The pulses may, in some examples, form a pattern intended to obtain a retinal response. Sensor 104 may capture responses from the retina of eye 114. Detection of responses by sensor 104 may be improved by positioning based on the visual guide from alignment system 118. The captured responses may form test data (e.g., ERG data). Physiological measurement system 116 may present the test data as a waveform through display 126. A caregiver may analyze the waveform to diagnose patient 112.

FIG. 1B illustrates another example measurement environment 130 in accordance with some aspects of the present disclosure. Example measurement environment 130, as illustrated, includes physiological measurement system 116, optical assembly 120, camera 124, display 126 and patient 112. Patient 112, as illustrated, is lying down. However, other options are also possible. Patient 112 may, for example, be sitting or standing.

Physiological measurement system 116, optical assembly 120, camera 124, and display 126 may include any of the features or perform any of the functionality described herein, at least with respect to FIG. 1A. Physiological measurement system 116 may, in some examples, be implemented on one or more computing devices. The one or more computing devices may, in some examples, communicate through a network using any suitable communication protocol. The network may, as one example, be a local network. However, as another example, the network may be a distributed network. The distributed network may include one or more computing devices spread across multiple locations or nodes. Suitable communication protocols may include, but are not limited to, transmission control protocol / internet protocol (TCP/IP), hypertext transfer protocol (HTTP), hypertext transfer protocol secure (HTTPS), message queueing telemetry transport (MQTT), constrained application protocol (CoAP), Bluetooth low energy (BLE), and the like, or some combination thereof.

Physiological measurement system 116, optical assembly 120, camera 124, and display 126 may, in some examples, be used to conduct a variety of testing on patient 112 relating to ocular conditions or other conditions affecting the visual pathway. One example, as described with respect to FIG. 1A, may be ERG testing. As another example, a pattern may be presented to patient 112 on a display. The display may, for example, be display 126. As another example, the patient 112 may view the pattern on one display, while a caregiver monitors the testing on another display. The pattern presented to patient 112 may change over time. Sensor 104 may capture responses from the retina of eye 114. The captured responses may form test data (e.g., ERG data). Physiological measurement system 116 may present the test data as a waveform through display 126. A caregiver may analyze the waveform to diagnose patient 112

Detection of responses by sensor 104 may be improved by positioning based on the visual guide from alignment system 118. Alignment system 118 may, for example, present a visual guide to a caregiver (e.g., through display 126) using any of the methods described with respect to FIG. 1A. The caregiver may adjust positioning of the sensor 104 based on the visual guide.

### Example Sensor Setups

FIG. 2A illustrates an example sensor setup of sensor 104, in accordance with some aspects of the disclosure. Sensor 104A, as illustrated, is placed inferior to (e.g., beneath) eye 114A. Sensor 104B as illustrated is placed inferior to eye 114B. A caregiver may, for example, place a sensor 104A such that an alignment portion 140 of the sensor 104A is inferior to the pupil of eye 114A based on a visual guide from alignment system 118, as described herein. In this placement, and with respect to the patient's nose, the alignment portion 140 is proximal and a second edge 142 of the sensor 104A is distal. The alignment process may be repeated for the patient's other eye 114B. Alignment of sensor 104 using alignment system 118 will be described further at least with respect to FIG. 4. While FIG. 2A illustrates sensors 104A and 104B placed inferior to eyes 114A and 114B, respectively, other placements may be used. A sensor 104 may, for example, be placed on an eye 114, superior to an eye 114, lateral to an eye 114, or in another location. Various positions of sensor 104 may be described further herein, at least with respect to FIG. 2B.

FIG. 2B illustrates various positions of sensor 104, which may be used in accordance with some aspects of the present disclosure. As illustrated, sensor 104 may be of any suitable size and shape. Sensor 104 may also be positioned in various locations proximate to eye 114. Alignment system 118 may guide positioning of sensor 104 using any of the methods described herein. This may advantageously improve the accuracy of data collected using the sensor 104 (e.g., during ERG testing).

Sensor setup 152, for example, includes a sensor 104 placed on eye 114. Sensor 104 of sensor setup 152, as illustrated, includes an eye-contacting arrangement with a posterior surface proximate to eye 114, and an anterior surface distal from eye 114. The posterior surface, as illustrated, is configured to contact the eye. The posterior surface may, in some examples, comprise one or more electrodes. The anterior surface may comprise light emitter 122. The anterior surface may additionally, or alternatively comprise an optical surface configured to, as an example, diffuse any illumination provided to the surface or maintain good image quality through the sensor 104. The alignment system 118 may guide positioning of or assess positioning quality with respect to sensor 104 onto eye 114 using any of the methods described herein, such as with respect to FIG. 1A-1B. Alignment system 118 may, for example, identify a pupil of eye 114 based on image data from a camera (e.g., camera 124 of FIG. 1A). Alignment system 118 may subsequently output a visual guide to a caregiver for positioning of the sensor 104 on eye 114. Alignment system 118 may, for example, output a visual guide through display 126 using any of the methods described with respect to FIG. 1A.

Sensor setup 154, as illustrated, also includes a sensor 104 placed on eye 114. Sensor 104 of sensor setup 154, as illustrated, includes a thread electrode to be placed at a preferred position inferior to the pupil. The preferred position may be, for example, on the cornea or deep within the conjunctival sac. Position 200 illustrates a position on the cornea, for example. Position 202 illustrates a position within the conjunctival sac, as another example. The alignment system 118 may guide positioning or assess positioning quality of sensor 104 onto eye 114 using any of the methods described herein.

Sensor setup 156, as illustrated, includes a sensor 104 placed superior to eye 114. Sensor 104 of sensor setup 156 may be configured to conform with skin of patient 112 above the eye 114. While one sensor 104 is illustrated in sensor setup 156, other configurations may be implemented. A different sensor 104 may be positioned superior to each eye of patient 112. Alignment system 118 may guide positioning or assess positioning quality of each sensor 104 using any of the methods described herein.

Sensor setup 158, as illustrated, includes a sensor 104 placed around eye 114. Sensor 104 of sensor setup 158, as illustrated, includes a combination of portions above eye 114, portions to the side of eye 114, and portions below eye 114. Other options are also possible. Sensor 104 may, in some examples, include portions above eye 114, portions to the side of eye 114, portions below eye 114, and the like, or any combination thereof. Sensor 104 of sensor setup 158 may further be configured to conform with skin of patient 112. While one sensor 104 is illustrated in sensor setup 156, other configurations may be implemented. A sensor 104 may be positioned around each eye of patient 112. Alignment system 118 may guide positioning or assess positioning quality of each sensor 104 using any of the methods described herein.

While sensor setup 152, sensor setup 154, sensor setup 156, and sensor setup 156 are illustrated in FIG. 2B, other options are also possible. Sensor 104 may alternatively be placed in regions including, but not limited to, over an eyelid of patient 112, on a fornix of eye 114, and the like, or some combination thereof.

### Example Visual Guide for Alignment of Sensor Position

FIG. 3A illustrates an example visual guide for alignment of sensor 104. The alignment system 118 may generate various user interfaces for alignment of sensor 104, for example, an identification display 304 and a sensor alignment display 306. Each of identification display 304, and sensor alignment display 306 may be output through a display accessible to a caregiver, such as display 126 of FIGS. 1A-1B.

Alignment system 118 may, in some examples, generate identification display 304. Identification display 304, as illustrated, includes a textual portion 314 and a pupil alignment circle 316, an image portion 318. The alignment system 118 may, for example, generate identification display 304 to begin an alignment process for positioning sensor 104 with respect to eye 114 (the patient's right eye in this example). Textual portion 314 includes instructions to “[a]lign pupil inside circle.” This language is exemplary. Other language may be used in some examples.

Image portion 318 includes image data for eye 114, surrounding facial features of eye 114, or some combination thereof. The image data may be captured using a camera (e.g., camera 124 of FIG. 1A). Image portion 318 may be updated in substantially real time as the camera 124 captures new image data. For example, image portion 318 may provide a substantially live video display of the field of view of the camera 124. Image portion 318 may accordingly include eye 114, surrounding facial features of eye 114, sensor 104, and the like, as they change over time. Alignment system 118 may superimpose alignment circle 316 over image portion 318. This may facilitate detection of the pupil for alignment of sensor 104 with respect to eye 114.

Based on the instructions of textual portion 314, the caregiver may adjust a device (e.g., device 132) with respect to eye 114. Movement of the device 132 may cause the position of eye 114, components of eye 114 (e.g., pupil, iris, eyelid, canthus of the eye, etc.), sensor 104, and the like, to move within image portion 318 responsive to updated image data from camera 124. The caregiver may use movement displayed within image portion 318 to align a pupil of eye 114 within alignment circle 316. In some examples, the caregiver may skip performing the test (e.g., ERG testing), including aligning the pupil of eye 114. The caregiver may alternatively indicate that they have aligned the pupil of eye 114 within alignment circle 316. Testing (e.g., ERG testing) may then continue. Alignment system 118 may then generate sensor alignment display 306.

Sensor alignment display 306, as illustrated, includes textual portion 326, and image portion 318. Alignment system 118 may update identification display 304 into sensor alignment display 306, in some examples. Alignment system 118 may generate pupil identifier 334 (e.g., a crosshair or other visual indicator superimposed over the image data) to further indicate a position of the pupil of eye 114. Knowledge of a position of the pupil of eye 114 may advantageously facilitate positioning of sensor 104 by a caregiver. Alignment system 118 may determine, based on identification of one or more features or components of eye 114, a location or range of locations at or within which sensor 104 should be placed. These locations may depend on the sensor type currently being used. Alignment system 118 may, for example, identify features or components of eye 114 after adjustment of a device (e.g., device 132) by a caregiver to align the pupil of eye 114 within alignment circle 316, as described above. Based on the determined features or components (e.g., pupil location, lower eyelid location), alignment system 118 may determine a position for sensor 104 or a particular portion of the sensor 104, such as a first edge that is nearest the patient's nose. The first edge may therefore be referred to as the alignment portion 140. The determined position for the alignment portion 140 may improve alignment of sensor 104 with respect to eye 114.

Alignment system 118 may generate a visual guide, such as graphical alignment zone 330, to indicate the determined position to the caregiver. Although the description herein provides a graphical alignment zone as the visual guide, the graphical alignment zone is used for purposes of illustration only and is not intended to be limiting, required, or exhaustive. In some embodiments, additional or alternative visual guides may be used. For example, a crosshair, arrow, shading, background blurring, or other visual effects may serve as visual guides for alignment of sensors 104 (or components thereof, such as alignment portion 140).

For example, display 126 may be a full-color display. Alignment zone 330 may, in further examples, be highlighted in a particular color, such as green. Other zones indicating misalignment may be highlighted in a different color, such as red. Other options are also possible. During dark-adapted testing for example, the display 126 may use red light. Accordingly, visual guides (e.g., alignment zone 330, zones indicating misalignment, etc.) may be of different tones of red, so as to not materially affect dark adaptation.

In the example illustrated in FIG. 3A, alignment system 118 superimposes alignment zone 330 over image data displayed in image portion 318. Textual portion 326 includes additional instructions regarding alignment of sensor 104 for positioning with respect to eye 114. Textual portion 326 includes, as illustrated, that sensor 104 must be “2 mm below eyelid,” with an alignment portion 140 (e.g., a first edge) of sensor 104 inside/below alignment zone 330. The instructions provided by textual portion 326 may, in some examples, generally describe the location of alignment zone 330 within image portion 318. A caregiver may activate a skip control (e.g., a button or menu item) to skip performing the test (e.g., ERG testing), including aligning the pupil of eye 114. A caregiver may additionally, or alternatively, activate a start control (e.g., a button or menu item) to begin testing visual function of eye 114. Activation of the start control may, for example, begin ERG testing of eye 114.

In some examples, an alignment system (e.g., alignment system 118) may further generate an input display. The input display may, for example, facilitate turning on or off features. The input display may, for example, facilitate turning off guided alignment, as described herein. The caregiver may in further examples place sensor 104 without generation of a visual guide by alignment system 118. The input display may, as another example, include a tested eye entry to indicate which eye (e.g., either the patient's left eye or right eye) is to be tested. A caregiver may determine to turn the feature off through the input display. However, the alignment system 118 may alternatively use image data to automatically determine whether the patient's right or left eye is to be tested based on detected features of the patient's face (e.g., the face of patient 112). Alignment system 118 may, for example, identify the location of the nose of patient 112 with respect to eye 114 using a computer vision algorithm to detect the presence and location of the nose of patient 112 in an image. Based on the relative positions of the nose of patient 112 and eye 114, alignment system 118 may determine which eye is being tested. Which eye is being tested may also be determined by the device stating which eye is to be tested, or by examining the image data overall or for specific anatomical landmarks such as the canthi of the eye.

In some examples, an alignment system (e.g., alignment system 118) may receive image data that includes at least part of the sensor in addition to the eye. The alignment system may use the image data to verify whether the sensor has been properly positioned. For example, the alignment zone is used not only as a visual guide regarding positioning of the sensor, but also to confirm that the sensor is actually positioned at the determined position to align the sensor for measuring a signal from the eye. During or after sensor placement, the alignment system obtains image data and compares the sensor's observed position with the preferred position. If the sensor is properly placed, the system can allow testing to proceed; if not, it can indicate misalignment (e.g., using visual and/or audible feedback), or even block continuation until the sensor is repositioned. This verification reduces instances in which the device would take measurements using the sensor when the sensor is outside the alignment zone, so the measurements providing a physiological measurement are obtained with more consistent positioning of the sensor relative to the eye of a patient. Improperly aligned sensors may be indicated as such. Alternatively, the alignment system may not have such a verification step.

Components illustrated with respect to FIG. 3A may, in some examples, be omitted, combined, or rearranged. Textual portion 314 may, for example, be omitted, have different wording, and the like, or some combination thereof. Textual portion 326 may, as another example, be omitted, have different wording, and the like, or some combination thereof.

FIG. 3B illustrates an example visual guide for alignment of sensor 104 as eye 114 moves over time. An alignment system (e.g., alignment system 118), may determine a position for alignment of sensor 104 with respect to eye 114. The alignment system 118 may determine the position based on identification of components of eye 114. To facilitate positioning of sensor 104 by a caregiver, alignment system 118 may superimpose an alignment zone 354 on an image displayed to the caregiver. Alignment zone 354 may indicate a region in which sensor 104 (or a portion thereof, such as alignment portion 140) would be substantially aligned, if positioned. Alignment system 118 may further generate misalignment zone 356. Misalignment zone 356 may indicate a region in which sensor 104 (or a portion thereof, such as alignment portion 140) would be substantially misaligned, if positioned. A misalignment zone may not indicate every unacceptable location. For example, it may indicate only the most likely unacceptable locations, so as not to clutter the display 126. Although the alignment zones (e.g., alignment zone 354) and misalignment zones (e.g., misalignment zone 356) may only be intended to specify the position of alignment portion 140, misalignment zones may only be present in regions that no portion of sensor 104 should be present in order to reduce the chance of caregiver confusion. Generation of alignment zone 354 and misalignment zone 356 may improve positioning of sensor 104 with respect to eye 114. This may improve a quality of data collected by sensor 104 during testing (e.g., ERG testing).

As described with respect to FIG. 3B, alignment system 118 may generate one or more alignment displays 340 (e.g., alignment display 306). The sensor alignment displays may be provided to the caregiver through display 126 of FIGS. 1A-1B. The alignment displays 340, as illustrated, change as eye 114, facial features surrounding eye 114, and the like, move over time. Alignment displays 340 may thus advantageously provide an indication of a proper position for sensor 104 and/or dynamic feedback regarding alignment of a position for sensor 104.

Alignment display 340A, for example, represents feedback regarding alignment of a position for sensor 104 with respect to eye 114 at time A. Alignment display 340B represents a visual guide relating to alignment of a position for sensor 104 with respect to eye 114 at time B. Alignment display 340C represents a visual guide relating to alignment of a position for sensor 104 with respect to eye 114 at time C. Alignment display 340D represents a visual guide relating to alignment of a position for sensor 104 with respect to eye 114 at time D. Sensor alignment display 340E represents a visual guide relating to a position for sensor 104 for alignment with respect to eye 114 at time E. Alignment display 340F represents a visual guide relating to alignment of a position for sensor 104 with respect to eye 114 at time F.

Alignment zone 354, as illustrated, shifts as eye 114 moves between alignment display 340A and alignment display 340B. Alignment system 118 may, for example, receive updated image data reflecting the position of eye 114 at time B. Alignment system 118 may identify the position of components of eye 114, facial features surrounding eye 114, and the like, based on the updated image data for time B. Alignment system 118 may accordingly determine a position at which sensor 104 is to be placed using any of the methods described herein. Alignment system 118 may adjust the position of alignment zone 354, misalignment zone 356, and the like based on the determined position. Alignment system 118 may repeat this process every time updated image data is received. Alignment system 118 may, for example, repeat this process at time C, time D, time E, time F, and the like, or some combination thereof.

By way of example, alignment system 118 may identify a position of upper eyelid 358. Alignment system 118 may consider this position in conjunction with components of eye 114 in determining a position for sensor 104 for alignment with respect to eye 114. The alignment system 118 may, for example, raise or lower the determined position at which sensor 104 is to be placed with respect to eye 114 based on the identified position for upper eyelid 358. The alignment system 118 may accordingly update a position of alignment zone 354, misalignment zone 356, and the like in alignment display 340C based on the determined position for sensor 104.

With respect to alignment display 340F, as another example, alignment system 118 may identify a position of upper eyelid 358. Alignment system 118 may further identify a position for lower eyelid 360. Alignment system 118 may consider the positions of upper eyelid 358, and lower eyelid 360, in conjunction with components of eye 114 in determining a position for sensor 104 for alignment with respect to eye 114. The alignment system 118 may, for example, raise or lower the determined position for sensor 104 with respect to eye 114 based on the identified position for upper eyelid 358. The alignment system 118 may, as another example, raise or lower the determined position for sensor 104 with respect to eye 114 based on the identified position for lower eyelid 360. The alignment system 118 may accordingly update a position of alignment zone 354, misalignment zone 356, and the like in alignment display 340F based on the determined position for sensor 104.

### Example Method for Alignment of Sensor Position

FIG. 4 illustrates an example computer-implemented method 400 for alignment of sensor position (e.g., sensor 104 position). Some of the processes, steps, and/or modules discussed herein with respect to FIG. 4 may be combined, separated into sub-parts, omitted entirely, and/or rearranged to run in a different order and/or in parallel. In addition, in some embodiments, different blocks may execute on various components of a single-device system (e.g., device 132 as shown in FIG. 1A which includes a physiological measurement system 116, optical assembly 120, camera 124, and display 126 integrated with or coupled to a single housing 136) or a multi-device system (e.g., physiological measurement system 116 in communication with an optical assembly 120, camera 124, and display 126 as shown in FIG. 1B).

At block 402, physiological measurement system 116 may access image data from a camera, the image data including an eye of a patient (e.g., eye 114 of patient 112). Physiological measurement system 116 may, in some examples, cause camera 124 to capture image data with respect to patient 112. As described with respect to FIG. 1A, a camera 124 may capture image data of an eye 114, facial features surrounding eye 114, part or all of sensor 104, and the like, or some combination thereof. Camera 124 may capture image data continuously, at regular intervals, at variable intervals, and the like, or some combination thereof. Physiological measurement system 116 may communicate with camera 124 to access the image data. Camera 124 may, in further examples, provide captured image data to physiological measurement system 116.

At block 404, physiological measurement system 116 may identify a feature of eye 114 based on the image data. Physiological measurement system 116 may, for example, use alignment system 118 to analyze the image data to identify of a component of eye 114. As one example, alignment system 118 may identify a pupil of eye 114. Based on the position of the pupil, alignment system 118 may determine a position for sensor 104, as described further with respect to block 406.

Alignment system 118 may identify additional or alternative features. Alignment system 118 may, for example, identify additional features of components of eye 114, surrounding facial features of eye 114, and the like, or some combination thereof. As described herein, features of eye components may include, but are not limited to, positions of the eyelids, position of the iris, position of the pupil, position of a canthus of eye 114, and the like, or some combination thereof. As described with respect to FIG. 3B, alignment system 118 may detect an upper eyelid 358, and a lower eyelid 360. Alignment system 118 may use the additionally detected features to refine a position for sensor 104.

As another example, alignment system 118 may identify a nose of patient 112. Alignment system 118 may, in some examples, determine which eye 114 of patient 112 is under analysis. Alignment system 118 may, for example, determine whether an eye 114 is a left eye or right eye based on identification of the nose of patient 112 or features of the eye 114 included in image data from camera 124. Alignment system 118 may additionally, or alternatively, use the identification of the nose in conjunction with other identified features of the eye 114 to refine a position for sensor 104.

At block 406, physiological measurement system 116 may determine a position at which a sensor 104 (or a portion thereof, such as alignment portion 140) is to be placed. Physiological measurement system 116 may, for example, determine a position for sensor 104 using alignment system 118. Positioning of sensor 104 for testing may, in some examples, align with one or more rules. The rules may, for example, set acceptable distances with respect to components of eye 114, surrounding facial features, and the like, or some combination thereof.

With respect to FIG. 3A, a rule may be that sensor 104 is to be at least or at most a threshold distance from an eye component (e.g., about 0.5 mm below an eyelid or pupil, about 1.0 mm below an eyelid or pupil, about 1.5 mm below an eyelid or pupil, about 2.0 mm below an eyelid or pupil, about 2.5 mm below an eyelid or pupil, about 3.0 mm below an eyelid or pupil, about 3.5 mm below an eyelid or pupil, or about 4.0 mm below an eyelid or pupil). Another rule may, for example, set a range (e.g., 0 mm- 4 mm) for positioning sensor 104 with respect to lower eyelid 360. Another rule may, for example, be that the alignment portion 140 of sensor 104 be directly below a center of a pupil, a center of an iris, midway between the canthi of eye 114, or an offset in the medial or lateral direction from any of these or other features identified in the image data. The rules may additionally, or alternatively, set acceptable distances with respect to facial features surrounding eye 114. The facial features surrounding eye 114 may include, but are not limited to, a nose of patient 112, an eyebrow of patient 112, and the like, or some combination thereof.

In some examples, alignment system 118 may determine a position at which sensor 104 is to be placed by determining zones to facilitate positioning of sensor 104. Alignment system 118 may, for example, determine an alignment zone (e.g., alignment zone 330 of FIG. 3A, alignment zone 354 of FIG. 3B, etc.) indicating a position for sensor 104. The alignment zone may, in some examples, correspond to one or more ranges included in the rules. For example, a vertical position of the alignment zone may be impacted by a rule relating to an acceptable range of distances with respect to an eyelid (e.g., upper eyelid 358, lower eyelid 360, etc.). The range may be, for example, 0 mm-4 mm. A horizontal position of the alignment zone may, as another example, be impacted by a rule relating to an acceptable range of distances with respect to a nose, a center of the pupil, a center of an iris, midway between the canthi of the eye, or an offset in the medial or lateral direction from any of these or other landmarks of the patient 112. Alignment system 118 may consider these rules in conjunction when generating the alignment zone.

At block 408, physiological measurement system 116 may provide a visual guide regarding the position of the sensor. Physiological measurement system 116 may provide the visual guide regarding the position of the sensor using alignment system 118. Alignment system 118 may, for example, generate a sensor alignment display (e.g., sensor alignment display 306 of FIG. 3A, alignment visual guide display 340 of FIG. 3B). As described with respect to FIGS. 3A-3B, alignment system 118 may superimpose an alignment zone over image data received from camera 124. The sensor alignment display may then be output to a caregiver through a display (e.g., display 126 of FIGS. 1A-1B).

The alignment system 118 may, in some examples, consider one or more rules impacting an alignment zone independent of identified locations for components of eye 114, surrounding facial features of eye 114, and the like, or some combination thereof. A rule may for example, set a minimum width for an alignment zone. The minimum width may, for example, be based on an expected ability of a caregiver to control the positioning of sensor 104 within an alignment zone. A caregiver may for example, struggle to position sensor 104 if a width of the alignment zone is too small A rule may accordingly set a range of acceptable locations that include the position identified at block 406. The range may account for an expected ability to control the positioning of sensor 104, a decrease in system performance for nonideal sensor positioning, and the like, or some combination thereof. The range may be, for example, less than or equal to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17, mm, 18 mm, 19 mm, or 20 mm wide. While the position identified in block 406 may be within an alignment zone, it may not be centered in the alignment zone. For example, an alignment zone may span from a center of a pupil medially by 3 mm and laterally by 4 mm, giving an entire range of less than or equal to 7 mm.

Alignment system 118 may, in some examples, also generate misalignment zones (e.g., misalignment zone 356 of FIG. 3B). Misalignment zones may indicate a zone in which sensor 104 would be misaligned, if positioned. The location of a misalignment zone may also be impacted by the rules. With respect to an example described above, a rule may indicate an acceptable range of distances with respect to lower eyelid is between 0 mm and 4 mm.

The range may be, as one example, be 2 mm to 4 mm. In further examples, distances outside this range include less than 2 mm to the lower eyelid and further than 4 mm from the lower eyelid. The alignment system 118 may accordingly generate a misalignment zone less than 2 mm from lower eyelid 360. The alignment system 118 may also generate a misalignment zone further than 4 mm from lower eyelid 360. Alignment system 118 may generate a visual guide regarding position of the sensor at block 408. The visual guide may include an alignment zone. The visual guide may further include a misalignment zone.

A rule may indicate an acceptable range of distances with respect to medial-lateral distances from the center of the pupil spanning from 3 mm medially and 4 mm laterally. Alignment system 118 may accordingly generate a misalignment zone immediately medial of the alignment zone. While the alignment system 118 may also generate a misalignment zone immediately lateral of the alignment zone, other parts of a properly-placed sensor 104 (e.g., not alignment portion 140), may be located lateral to the alignment zone. In these cases, having a lateral misalignment zone may cause confusion for caregivers that forget the alignment aids are for the alignment portion 140.

The alignment system 118 may, in some examples, further superimpose one or more misalignment zones onto the sensor alignment display. With reference to FIG. 3B, alignment system 118 may superimpose a misalignment zone 356 between alignment zone 354 and a nose of patient 112.

The alignment system 118 may, in some examples, update the sensor alignment display. With reference to FIG. 3B, the alignment system 118 may update the position of an alignment zone 354 and misalignment zone 356 over time as eye 114 and facial features surrounding eye 114 move.

At block 410, physiological measurement system 116 may take measurements. A caregiver may, for example, position sensor 104 or check the position of sensor 104 based on the provided visual guide at block 408. A misaligned sensor 104 may be corrected by a caregiver before the start of measurement in block 410 Physiological measurement system 116 may subsequently cause an optical assembly (e.g., optical assembly 120 of FIG. 1A) to conduct testing on eye 114 (e.g., ERG testing). Optical assembly 120 may, for example, use light emitter 122 to emit light that reaches eye 114 using any of the methods described with respect to FIG. 1A. Light may, for example, reach eye 114 if emitted into eye 114, through an eyelid, and the like, or some combination thereof. Sensor 104 may capture electrical responses from a retina of eye 114. Physiological measurement system 116 may generate a waveform representing the measured responses. Physiological measurement system 116 may further present the generated waveform to the caregiver (e.g., through display 126 of FIGS. 1A-1B).

### Example Hardware for an Alignment System

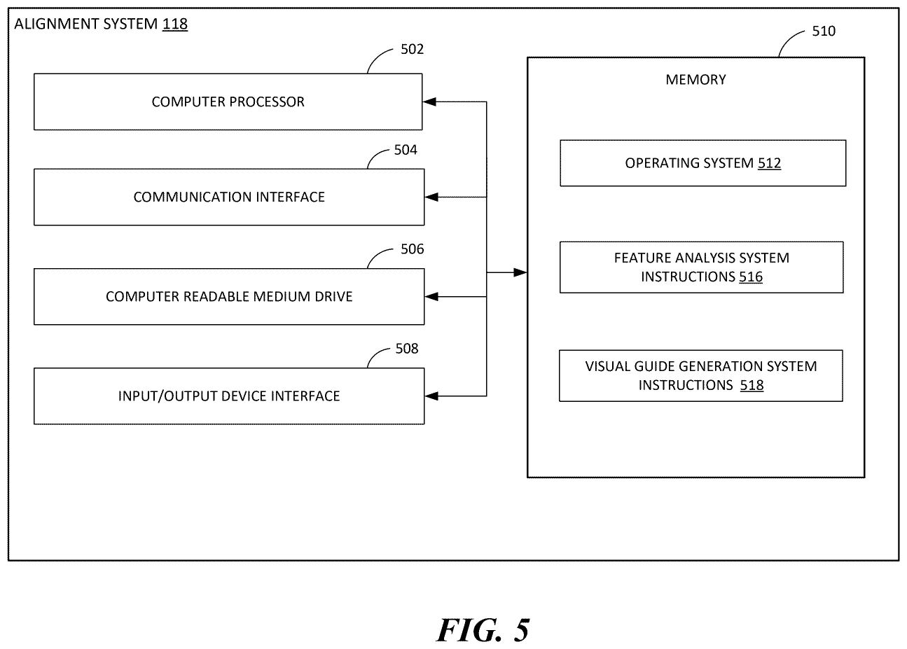

FIG. 5 illustrates a general architecture for an alignment system 118 including an arrangement of computer hardware and software that may be used to implement aspects of the present disclosure. The hardware may be implemented on physical electronic devices, as described in greater detail below. The alignment system 118 may include more (or fewer) elements than those shown in FIG. 5. Additionally, the general architecture stated in FIG. 5 may be used to implement one or more other components illustrated in FIG. 1A. The general architecture stated in FIG. 5 may, as another example, be used to implement physiological measurement system 116.

As illustrated, the alignment system 118 includes a computer processor 502, a communication interface 504, a computer-readable medium drive 506, an input/output device interface 508, and a memory 510, all of which may communicate with one another by way of a communication bus. The communication interface 504 may provide connectivity to one or more networks or computing systems. The computer processor 502 may thus receive information and instructions from other computing systems or services, such as a user computing device. The computer processor 502 may also communicate to and from memory 510 and further provide output information for a display (not shown) via the input/output device interface 508. The input/output device interface 508 may also accept input from an optional input device (not shown).

The computer-readable medium drive 506 may be used in storing data and code. Illustratively, the computer-readable medium drive 506 may be a non-transitory medium that stores data and code (e.g., a hard drive, an SD card, EEPROM, etc.). In some examples, the computer-readable medium drive 506 may be an external hard drive communicatively coupled to the alignment system 118 (e.g., through a cable or wirelessly). Alternatively, the computer-readable medium drive 506 may be internal to the alignment system 118. In some examples, the computer-readable medium drive 506 may be used to carry out any of the functions described below with respect to memory 510.

The memory 510 may store an operating system 512 that provides computer program instructions for use by the computer processor 502 in the general administration and operation of the alignment system 118. Memory 510 may further include computer program instructions and other information for implementing aspects of the present disclosure. For example, the memory 510, as illustrated includes feature analysis system instructions 516 and visual guide generation system instructions 518. Alignment system 118 may execute feature analysis system instructions 516 to determine a location of one or more components of an eye (e.g., eye 114), a location of facial features surrounding eye 114, and the like, or some combination thereof.

Alignment system 118 may execute visual guide generation system instructions 518 to generate a visual guide with respect to a position of a sensor (e.g., sensor 104) for alignment with respect to eye 114. Alignment system 118 may, for example, execute visual guide generation system instructions 518 to generate an alignment zone, a misalignment zone, and the like or some combination thereof. Alignment system 118 may further execute visual guide generation system instructions 518 to generate sensor alignment displays (e.g., sensor alignment display 306 of FIG. 3A, sensor alignment display 340 of FIG. 3B, etc.).

### EXAMPLE EMBODIMENTS

Examples of some embodiments of the present disclosure are described in view of the following clause.

Clause 1. A device comprising: a light emitter; a camera; and one or more processors programmed by executable instructions to: access image data from the camera, the image data representing an eye of a patient; determine a position at which a sensor should be placed based on the image data, the position determined so as to align the sensor for measuring a signal from the eye; generate a visual guide regarding positioning of the sensor, the visual guide comprising an alignment zone that includes the determined position; cause the light emitter to emit light that reaches the eye; and take measurements using the sensor, the measurements providing a physiological measurement.

Clause 2. The device of claim 1, wherein the signal is at least one of an electrical signal, an optical signal, a thermal signal, a mechanical signal, or a magnetic signal.

Clause 3. The device of claim 1, wherein the signal is an electrical signal.

Clause 4. The device of any of claims 1-3, further comprising the sensor.

Clause 5. The device of any of claims 1-4, wherein the one or more processors are further programmed by the executable instructions to verify that the sensor has been positioned in the alignment zone.

Clause 6. The device of any of claims 1-5, wherein the image data further represents at least part of the sensor.

Clause 7. The device of any of claims 1-6, wherein the one or more processors are further programmed by the executable instructions to identify a component of the eye based on the image data, wherein the position at which the sensor is to be placed is determined based on the identified component of the eye.

Clause 8. The device of claim 7, wherein the one or more processors are further programmed by the executable instructions to generate the alignment zone based on one or more rules, the one or more rules comprising a rule to position the sensor within a range of distances from the identified component of the eye.

Clause 9. The device of any of claim 7-8, wherein the component of the eye comprises at least one of a pupil of the eye, an iris of the eye, an upper eyelid of the eye, a lower eyelid of the eye, or a canthus of the eye.

Clause 10. The device of claim 9, wherein the alignment zone is limited in extent in a medial-lateral direction to a span that includes a center of the pupil.

Clause 11. The device of claim 9, wherein the alignment zone is limited in extent in a superior-inferior direction to a span that includes an offset from the lower eyelid.

Clause 12. The device of claim 9, wherein the alignment zone comprises a band in a superior-inferior direction that includes a center of the pupil.

Clause 13. The device of claim 9, wherein the alignment zone comprises a band in a medial-lateral direction offset from the lower eyelid.

Clause 14. The device of claim 9, wherein the alignment zone is limited in extent in a medial-lateral direction to a first span that includes a center of the pupil and is limited in extent in a superior-inferior direction to a second span that includes an offset from the lower eyelid.

Clause 15. The device of any of claims 1-14, wherein the image data further represents a surrounding facial feature.

Clause 16. The device of claim 15, wherein the surrounding facial feature comprises a nose.

Clause 17. The device of any of claims 1-16, wherein the one or more processors are further programmed by the executable instructions to: access updated image data from the camera, the updated image data representing the eye of a patient at time subsequent to a time of the image data; determine an updated position at which the sensor should be placed based on the updated image data, the updated position determined so as to align the sensor for measuring the signal; and provide an updated visual guide regarding updated positioning of the sensor, the updated visual guide comprising an updated alignment zone including the updated position.

Clause 18. The device of claim 17, wherein the one or more processors are further programmed by the executable instructions to identify a component of the eye based on the updated image data, wherein the updated position is determined based on the identified component of the eye.

Clause 19. The device of any of claims 17-18, wherein the updated position at which the sensor should be placed is provided in real time.

Clause 20. The device of any of claims 1-19, wherein the one or more processors are further programmed by the executable instructions to generate a misalignment zone, the misalignment zone indicating a second range of distances within which the sensor would be misaligned, if placed.

Clause 21. The device of any of claims 1-20, wherein the determined position for the sensor is at least one of inferior to the eye, superior to the eye, proximate to the eye, or on the eye.

Clause 22. The device of any of claims 1-21, wherein the sensor is detachable.

Clause 23. The device of any of claims 1-22, wherein, to take measurements using the sensor, the one or more processors are further programmed by executable instructions to begin performing electroretinography (ERG) testing.

Clause 24. A computer-implemented method comprising: as performed by a computing system comprising one or more computer processors programmed to execute specific instructions, accessing image data from a camera, the image data representing an eye of a patient; determining a position at which a sensor should be placed based on the image data, the position determined so as to align the sensor for measuring a signal from the eye; generating a visual guide regarding positioning of the sensor, the visual guide comprising an alignment zone including the determined position; and taking measurements using the sensor, the measurements providing a physiological measurement.

Clause 25. The computer-implemented method of claim 24, wherein the signal is at least one of an electrical signal, an optical signal, a thermal signal, a mechanical signal, or a magnetic signal.

Clause 26. The computer-implemented method of claim 24, wherein the signal is an electrical signal.

Clause 27. The computer-implemented method of any of claims 24-26, further comprising identifying a component of the eye based on the image data, wherein the position at which the sensor should be placed is determined based on the identified component of the eye.

Clause 28. The computer-implemented method of claim 27, further comprising generating the alignment zone based on one or more rules, the one or more rules comprising a rule to position the sensor within a range of distances from the identified component of the eye.

Clause 29. The computer-implemented method of any of claims 27-28, wherein the component of the eye comprises at least one of a pupil of the eye, an iris of the eye, a lower eyelid of the eye, an upper eyelid of the eye, or a canthus of the eye.

Clause 30. The computer-implemented method of claim 29, wherein generating the visual guide comprises limiting the alignment zone in extent in a medial-lateral direction to a span that includes a center of the pupil.

Clause 31. The computer-implemented method of claim 29, wherein generating the visual guide comprises limiting the alignment in extent in a superior-inferior direction to a span that includes an offset from the lower eyelid.

Clause 32. The computer-implemented method of claim 29, wherein generating the visual guide comprises limiting the alignment zone in extent in a medial-lateral direction to a first span that includes a center of the pupil, and limiting the alignment in extent in a superior-inferior direction to a second span that includes an offset from the lower eyelid.

Clause 33. The computer-implemented method of any of claims 24-32, further comprising identifying a surrounding facial feature of the eye.

Clause 34. The computer-implemented method of any of claims 24-33, further comprising: accessing updated image data from the camera, the updated image data representing the eye of the patient; determining an updated position for the sensor based on the updated image data, the updated position configured to align the sensor for measuring the signal; and providing an updated visual guide regarding updated positioning of the sensor, the updated visual guide comprising an updated alignment zone including the updated determined position.

Clause 35. The computer-implemented method of claim 34, wherein the updated position at which the sensor is to be placed is provided in real time.

Clause 36. The computer-implemented method of any of claims 34-35, further comprising identifying a component of the eye based on the updated image data, wherein the updated position is determined based on the identified component of the eye.

Clause 37. The computer-implemented method of any of claims 24-36, further comprising generating a misalignment zone, the misalignment zone indicating a second range of distances within which the sensor would be misaligned, if placed.

Clause 38. The computer-implemented method of any of claims 24-37, wherein the determined position for the sensor is at least one of inferior to the eye, superior to the eye, proximate to the eye, or on the eye.

Clause 39. The computer-implemented method of any of claims 24-38, wherein, taking measurements using the sensor comprises beginning performance of electroretinography (ERG) testing.

Clause 40. One or more non-transitory computer-readable media storing instructions that, when executed, cause a computing system to perform operations comprising: accessing image data from a camera, the image data representing an eye of a patient; determining a position at which a sensor should be placed based on the image data, the position determined so as to align the sensor for measuring a signal from the eye; generating a visual guide regarding positioning of the sensor, the visual guide comprising an alignment zone including the determined position; and taking measurements using the sensor, the measurements providing a physiological measurement.

Clause 41. The one or more non-transitory computer-readable media of claim 40, wherein the instructions, when executed, cause a computing system to perform operations comprising identifying a component of the eye based on the image data, wherein the position at which the sensor should be placed is determined based on the identified component of the eye.

Clause 42. The one or more non-transitory computer-readable media of claim 40, wherein the instructions, when executed, cause a computing system to perform operations comprising: accessing updated image data from the camera, the updated image data representing the eye of the patient; determining an updated position for the sensor based on the updated image data, the updated position configured to align the sensor for measuring the signal; and providing an updated visual guide regarding the updated position of the sensor, the updated visual guide comprising an updated alignment zone including the updated determined position.

Clause 43. The one or more non-transitory computer-readable media of claim 42, wherein the instructions, when executed, cause a computing system to perform operations comprising identifying a component of the eye based on the updated image data, wherein the updated position is determined based on the identified component of the eye.

Clause 44. A device providing an indication of visual system function of a patient comprising: a light emitter; an optical assembly arranged so that light emitted from the light emitter reaches an eye of the patient; a camera arranged to image both the eye of the patient and an intended position for a sensor adapted to be applied and removed from the patient; and a controller that modulates a light emission from the light emitter to create a light stimulus and receives and analyzes an electrical signal from a visual system of the patient via the sensor, and provides the indication of visual system function based on that analysis, wherein the device is arranged to be used by a caregiver, and wherein the controller provides a visual guide to the caregiver regarding placement of the sensor based on images from the camera.

Clause 45. The device of claim 44, further comprising the sensor.

Clause 46. The device of any of claims 44-45, wherein the intended position for the sensor is on skin near the eye.

Clause 47. The device of any of claims 44-46, wherein the intended position for the sensor is below the eye.

Clause 48. The device of any of claims 44-47, wherein the visual guide is based in part on a location of a pupil of the eye.

Clause 49. The device of any of claims 44-48, wherein the visual guide is based in part on a location of the sensor.

Clause 50. The device of any of claims 44-49, wherein the visual guide is based in part on a location of a lower eyelid.

### Terminology and Other Considerations

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some examples, the computer system may be a cloud-based computing system whose processing resources are shared by multiple distinct business entities or other users.

Depending on the example, certain acts, events, or functions of any of the processes or algorithms described herein can be performed in a different sequence, can be added, merged, or left out altogether (e.g., not all described operations or events are necessary for the practice of the algorithm). Moreover, in certain examples, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the examples disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the examples disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another example, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the examples disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements and/or steps. Thus, such conditional language is not generally intended to imply that features, elements, and/or steps are in any way required for one or more examples or that one or more examples necessarily include logic for deciding, with or without other input or prompting, whether these features, elements and/or steps are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. Unless otherwise explicitly stated, the terms “set” and “collection” should generally be interpreted to include one or more described items throughout this application. Accordingly, phrases such as “a set of devices configured to” or “a collection of devices configured to” are intended to include one or more recited devices. Such one or more recited devices can also be collectively configured to carry out the stated recitations. For example, “a set of servers configured to carry out recitations A, B and C” can include a first server configured to carry out recitation A working in conjunction with a second server configured to carry out recitations B and C.

While the above detailed description has shown, described, and pointed out novel features as applied to various examples, it can be understood that various omissions, substitutions, and changes in the form and details of the devices or algorithms illustrated can be made without departing from the spirit of the disclosure. As can be recognized, certain examples described herein can be embodied within a form that does not provide all of the features and benefits set forth herein, as some features can be used or practiced separately from others. The scope of certain examples disclosed herein is indicated by the appended claims rather than by the foregoing description. All changes which come within the meaning and range of equivalency of the claims are to be embraced within their scope.

What is claimed is:

1. A device comprising:
a light emitter;
a camera; and
one or more processors programmed by executable instructions to:
access image data from the camera, the image data representing an eye of a patient;
determine a position at which a sensor should be placed based on the image data, the position determined so as to align the sensor for measuring a signal from the eye;
generate a visual guide regarding positioning of the sensor, the visual guide comprising an alignment zone that includes the determined position;
cause the light emitter to emit light that reaches the eye; and
take measurements using the sensor, the measurements providing a physiological measurement.

2. The device of claim 1, wherein the signal is at least one of an electrical signal, an optical signal, a thermal signal, a mechanical signal, or a magnetic signal.

3. The device of claim 1, wherein the signal is an electrical signal.

4. The device of claim 1, further comprising the sensor.

5. The device of claim 1, wherein the one or more processors are further programmed by the executable instructions to verify that the sensor has been positioned in the alignment zone.

6. The device of claim 1, wherein the image data further represents at least part of the sensor.

7. The device of claim 1, wherein the one or more processors are further programmed by the executable instructions to identify a component of the eye based on the image data, wherein the position at which the sensor is to be placed is determined based on the identified component of the eye.

8. The device of claim 7, wherein the one or more processors are further programmed by the executable instructions to generate the alignment zone based on one or more rules, the one or more rules comprising a rule to position the sensor within a range of distances from the identified component of the eye.

9. The device of claim 7, wherein the component of the eye comprises at least one of a pupil of the eye, an iris of the eye, an upper eyelid of the eye, a lower eyelid of the eye, or a canthus of the eye.

10. The device of claim 9, wherein the alignment zone is limited in extent in a medial-lateral direction to a span that includes a center of the pupil.

11. The device of claim 9, wherein the alignment zone is limited in extent in a superior-inferior direction to a span that includes an offset from the lower eyelid.

12. The device of claim 9, wherein the alignment zone comprises a band in a superior -inferior direction that includes a center of the pupil.

13. The device of claim 9, wherein the alignment zone comprises a band in a medial-lateral direction offset from the lower eyelid.

14. The device of claim 9, wherein the alignment zone is limited in extent in a medial-lateral direction to a first span that includes a center of the pupil and is limited in extent in a superior-inferior direction to a second span that includes an offset from the lower eyelid.

15. The device of claim 1, wherein the image data further represents a surrounding facial feature.

16. The device of claim 15, wherein the surrounding facial feature comprises a nose.

17. The device of claim 1, wherein the one or more processors are further programmed by the executable instructions to:
access updated image data from the camera, the updated image data representing the eye of a patient at time subsequent to a time of the image data;
determine an updated position at which the sensor should be placed based on the updated image data, the updated position determined so as to align the sensor for measuring the signal; and
provide an updated visual guide regarding updated positioning of the sensor, the updated visual guide comprising an updated alignment zone including the updated position.

18. The device of claim 17, wherein the one or more processors are further programmed by the executable instructions to identify a component of the eye based on the updated image data, wherein the updated position is determined based on the identified component of the eye.

19. The device of claim 17, wherein the updated position at which the sensor should be placed is provided in real time.

20. The device of claim 1, wherein the one or more processors are further programmed by the executable instructions to generate a misalignment zone, the misalignment zone indicating a second range of distances within which the sensor would be misaligned, if placed.

21. The device of claim 1, wherein the determined position for the sensor is at least one of inferior to the eye, superior to the eye, proximate to the eye, or on the eye.

22. The device of claim 1, wherein, to take measurements using the sensor, the one or more processors are further programmed by executable instructions to begin performing electroretinography (ERG) testing.
